(19) United States
(12) Patent Application Publication
(10) Pub. No.: US 2004/0234604 A1
(43) Pub. Date: Nov. 25, 2004

(54) Medical-technology product, process for its production, and use

(76) Inventors: Stefan Mecking, Freiburg (DE); Joerg C. Tiller, Freiburg (DE); Erich Odermatt, Schaffhausen (CH)

Correspondence Address: Nath & Associates, Sixth Floor, 1030 15th Street, N.W., Washington, DC, 20005, US

(21) Appl. No.: 10/714,552

(22) Filed: Nov. 17, 2003

(30) Foreign Application Priority Data
May 19, 2003 (DE) 103 23 597.3

(51) Int. Cl. A61K 9/14; A61K 33/24

(52) U.S. Cl. 424/486; 424/617

(57) ABSTRACT

The invention relates to a medical-technology product with a layer of a hybrid complex material composed of a branched amphiphilic macromolecule and of a metal nanoparticle, to the se of a hybrid complex material composed of a branched amphiphilic macromolecule and of a metal nanoparticle as a biocide for medical-technology products, and also to a process for producing a hybrid complex material composed of a branched amphiphilic macromolecule and of a metal nanoparticle, by dissolving a metal compound, with complexing, followed by reduction of the metal compound, and to a process for producing medical-technology products wherein the hybrid complex material is applied from outside to the product or is added to the polymer material of the product during its production.

### BACKGROUND OF THE INVENTION

1. Field of the Invention

The invention relates to a medical-technology product with a layer of a hybrid complex material, to the use of a hybrid complex material as biocide for medical-technology products, and also to a process for producing the hybrid complex material, and to a process for producing medical-technology products with the hybrid complex material.

2. Description of the Related Art

One of the greatest challenges in the field of everyday hygiene, and especially in the field of medicine, in particular during surgery, is the colonization of surfaces by health-threatening and undesirable colonies of microorganisms. Much interest is therefore directed towards preventing causes of infection from reaching, during and after an investigation or surgery, medical-technology products present for a relatively long or relatively short time within the interior of the body of a human or of an animal. Because the body temperature of 37° C. provides ideal growth conditions for microorganisms, ideal requirements for the colonization of surfaces are especially present in the case of implants, which remain long-term within the interior of the body. Very many post-operative complications currently arising in the form of infections are attributable to colonies of microorganisms on foreign body surfaces introduced for a short or long period into the body in the context of a medical investigation or surgery. Initial sterility of these medical-technology products cannot prevent subsequent colonization by microorganisms on the surface, and experiments have therefore been undertaken with the aim of providing biocidic properties through treatment of the materials used or coating of their surfaces. There are various known coatings based on the slow release of toxic agents. However, the release of these toxic agents is often associated with undesired side-effects for the organism. Attempts are therefore being made to provide systems which can prevent the microbial colonization of surfaces without the release of toxic biocides, and which are not attended by any undesirable allergical or toxic side-effects for the human organism.

Silver is known as one of the most toxic metals for microorganisms. Silver has an extremely wide-ranging antimicrobial profile, encompassing Gram-negative as well as Gram-positive bacteria. The microbial toxicity results from an attack of the silver ions on the trans-membrane-energy metabolism, and, apart from a few exceptions, provides high toxicity for the vast majority of microorganisms. In contrast, the human body can tolerate silver at concentrations of up to about 1 mg per day and person, and has no allergic reaction to silver. Silver colloids have long been known to have antimicrobial properties and at the same time to be relatively environmentally compatible and non-toxic (Biochemische Zeitschrift 1919, 94, 47). Various methods have therefore been tried for applying or incorporating silver or silver ions onto products requiring biocidic treatment.

For example, it is possible for products composed of a very wide variety of materials to be provided with a fine layer of metallic silver through plasma-assisted silver coating, or by means of IBAD processes (ion-beam-assisted deposition) in a vacuum vapour-deposition apparatus.

The invention is based on the object of providing medical-technology products with a long-term-active non-toxic biocidic coating which is active against intra- or post-operative microbial contamination and permits temporary or long-lasting use, without complications, within the body. This coating is to be easy to apply at the desired sites.

### SUMMARY OF THE INVENTION

This object is achieved through a medical-technology product with a layer of a hybrid complex material composed of a branched amphiphilic macromolecule and of a metal nanoparticle, the layer having been provided at least on the surface and at least on a portion of the surface.

The advantage of the inventive medical-technology product is in particular that the biocidic treatment, in particular coating, has adequately stable adhesion due to sources of interaction with the surface of the product materials, thus inhibiting release, i.e. removal of the biocidic material by wiping or washing. The products thus treated retain effective protection from microbial colonization, even after introduction into the body, or after a relatively short or relatively long post-operative presence within the body. Coating materials of this type are known from the publication by Mecking et al. (Chem. Comm. 2002, 3018-3019 of 19 Nov. 2002), the content of which is incorporated herein by way of reference. The complex-forming organic chemicals on which these hybrid complexes are based are known from US Patent U.S. Pat. No. 3,425,549 of 1969, and are of great importance as chelating reagents in the chemical industry.

The inventive medical-technology product has a layer of a hybrid complex material which is composed of a branched amphiphilic macromolecule and of a metal nanoparticle. This layer has been provided at least on the surface of the product, and on this surface at least on one portion of the entire surface of the product. The hybrid complex material may be present not only at the coated surface but also within the material of the product. The metal nanoparticles are not ionic particles, but are elemental metallic nanoparticles.

In one embodiment, each nanoparticle is surrounded by at least one branched amphiphilic macromolecule. The at least one macromolecule here encloses the metal nanoparticle on all sides in the manner of a capsule. It is also possible for a large number of individual macromolecules to encapsulate the metal nanoparticle.

The amphiphilic macromolecule is advantageously an amphiphilic polyalkyleneimine, in particular a polyethyleneimine or polypropyleneimine. It is also possible to use other alkyleneimines whose branched underlying structure has an adequate number of primary, secondary or tertiary nitrogen atoms to provide adequately stable encapsulation of the metal nanoparticle located in the interior.

In one particular embodiment, the degree of branching of the polyalkyleneimine is from 20 to 90%, preferably from 40 to 80%, in particular about 60%.

In another embodiment, the polyalkyleneimine has alkyl-substituted secondary or tertiary amino groups. The secondary or tertiary amino groups preferably bear methyl substituents or ethyl substituents.

The branched amphiphilic polyalkyleneimine advantageously has amide groups in particular oriented away from the metal nanoparticle in the interior of the polyalkyleneimine. In these amide groups, the N atoms derive from the polyalkyleneimine skeleton, and the carbon atoms derive from a carboxylic acid.

In one embodiment, the amide groups bear an aliphatic fatty acid radical, preferably oriented towards the outside. The number of carbon atoms in this fatty acid radical is from 6 to 22, preferably 12 to 18, and in particular 16, carbon atoms. The aliphatic radical of the fatty acid may be composed of branched or of unbranched carbon chains. It may moreover come either from saturated or else from at least partially unsaturated fatty acids. They are preferably linear, unsaturated, and have an even number of carbon atoms. This preferred orientation of the acid radical bonded by way of the amide group to the polyalkylene skeleton, and also the orientation of the amine groups of the polyalkyleneimine system towards the inside, gives this macromolecule its amphiphilic character. The hydrophobic outer side having the aliphatic radicals permits good adhesion to hydrophobic materials, in particular surfaces, of the medical-technology products. At the same time, the polar character of the amine groups in the interior of the macromolecule leads to enclosure of the metal nanoparticle, thus ensuring that there can be no repellent reactions with respect to hydrophobic surfaces of materials.

The amidation of the fundamental polyalkyleneimine structure can be achieved by way of various reagents and is known from the publication by Rannard and Davies (Org. Lett. 2000, 2, 2177). Preparation of the underlying polyalkyleneimine structure has previously been described in U.S. Pat. No. 3,425,549 for specific alkyleneimines and is known from U.S. Pat. No. 2,182,306 for polyethyleneimine, for example.

In one embodiment, the molecular weight of the macromolecule is from 800 to 20 000, preferably from 2 000 to 10 000 and in particular about 5 000. The molecular weight depends in particular on the number of carbon atoms, and also on the fatty acid radicals of the amide groups, and also on the number of carbon atoms in the alkyl radicals of the polyalkyleneimine, and on the degree of branching of the polyalkyleneimine. It seems that polyethyleneimines having relatively short fatty acid radicals and no alkyl substituents have relatively low molecular weight, whereas molecules having long-chain alkyl radicals and fatty acid radicals have a high molecular weight.

The metal nanoparticle is advantageously a silver nanoparticle or a copper nanoparticle, in particular silver. Silver, and also to a lesser extent copper, are the most toxic metals with respect to the following microorganisms from which protection is required: Gram-positive cocci, multiresistant coagulase-positive and -negative staphylococci and enterococci, Gram-negative enterobacteria, such as P. aeruginosa and C. albicans.

The ratio of silver atoms to the, preferably secondary or tertiary, nitrogen atoms in direct contact with them and in particular oriented towards the inside within the macromolecule is from 1:2 to 1:10, preferably from 1:3 to 1:5 and in particular 1:4. If the proportion of nitrogen atoms is too low, the number of these is insufficient to provide complete encapsulation around the silver atoms or silver nanoparticles, and either the amount of silver included by the macromolecules is very small or complete encapsulation of the silver atoms becomes impossible. In contrast, a large number of nitrogen atoms in direct contact with the silver atoms has no adverse affect on the properties, in particular stability, of the hybrid complex.

In one embodiment, the diameter of the hybrid complex is from 0.5 to 10 nm, preferably from 1 to 5 nm and in particular about 2 nm. The size of the amphiphilic hybrid complex is therefore towards the lower end of the range for currently known metal nanoparticles.

In an embodiment, the inventive product is a temporary or long-lasting implant for the body of a human or of an animal. These implants provided with the hybrid complex are preferably joint implants, stents, screws, nails, and plates for the repair of fractures, composed of metal and/or plastic, and are in particular hernia meshes and vessel prostheses, or else membranes and films, e.g. for adhesion prophylaxes, incontinence tapes, and textile implants generally. The biocidic coating of these implants permits their introduction even into acutely infected or infection-threatened regions of the body, because the implants themselves have antimicrobial action by virtue of the hybrid complex material, and actively contribute to the reduction of any existing or potential infection.

In another embodiment, the medical-technology products are medical instruments, in particular surgical scissors, forceps, and clips, or else catheters or probes, and other instruments in particular for minimally invasive microsurgery. Specifically in the case of these instruments exposed to mechanical stress, in particular through friction and wiping, the property of adhesion of the hybrid complex material to the surfaces is highly important, as is insolubility in an aqueous environment. As a result, even in the case of prolonged surgery or limited opportunity for sterilization of instruments to be used in an intervention, the risk of infection through the use of multiple-use instruments is advantageously low, in particular with respect to Creutzfeldt-Jakob or the problems posed by HIV.

The medical-technology products may also be products such as drainage tubes or suture material, these representing an intermediate group of medical-technology products between medical instruments and implants. Products such as wound dressings belong to this group.

In one embodiment, the medical-technology products have been produced from metal, preferably from titanium or from surgical steel.

In another embodiment, the material of the products is non-resorbable or at least to some extent resorbable polymers. In particular in the case of polymeric materials, the hybrid complex material may, besides a coating on the surface, also be present as a component added to the polymer material in the interior of the product.

In another embodiment, the material of the medical-technology products may also be ceramic.

The product is advantageously sterilizable and is available in particular in sterilized form. Sterilization methods which may be used are any of the currently available methods which do not alter either the chemical structure or the properties of the hybrid complex. The inventive medical-technology product is in sterile form when used. By virtue of the biocidic application, the medical-technology products thus coated may also be provided and opened prior to immediate use or implantation.

The invention also encompasses the use of a hybrid complex material composed of a branched amphiphilic macromolecule and of a metal nanoparticle as a biocide in medical-technology products. In the inventive hybrid complex material each metal nanoparticle in particular has been surrounded in the manner of a capsule by at least one branched amphiphilic macromolecule.

In one embodiment, the biocide has been applied to at least a portion of the surface of the medical-technology product. Depending on the use and field of use of the product, it can be advisable to provide the biocide only on one portion of the product which comes into contact with, preferably the interior of, the human body.

In another embodiment, the hybrid complex material has been incorporated directly into the interior of the medical-technology product.

It is also possible for the hybrid complex material to have been applied and, respectively, incorporated both on at least one portion of the surface and also in the interior of the medical-technology product. In one particular embodiment, the hybrid complex material may initially be present in the interior of the product and, in the case of resorbable products, a fresh product surface may be continuously exposed, thus exposing an undepleted biocidic layer which permanently protects the implant from microbial colonization until it has been completely resorbed.

The invention also encompasses a process for producing a hybrid complex material composed of a branched amphiphilic macromolecule and of a metal nanoparticle, where in particular each metal nanoparticle is encapsulated by at least one branched amphiphilic macromolecule. In the inventive process, a metal compound is dissolved, with complexing, in a solution of an amphiphilic polyalkyleneimine, in particular in an organic solvent.

The metal compound is preferably a silver salt, in particular silver nitrate. However, it is also possible to use other silver salts, in particular silver acetate, or copper salts, the toxic action of copper with regard to undesirable microorganisms being weaker than the action of silver.

After the metal compound has been dissolved, a reduction of the same compound takes place by means of a suitable reducing agent or of a combination of reducing agents, in particular using lithium borohydride/sodium thiosulphate. The solvent of the amphiphilic polyalkyleneimine is advantageously an aprotic, preferably aromatic, solvent. In one particular embodiment, the solvent is toluene.

In another embodiment, the metal compound may also be a metal complex, in particular a silver complex, which has lower stability than the complex with the polyalkyleneimine.

In one embodiment, use is made of an amphiphilic polyalkyleneimine which is produced via amidation of a branched polyalkyleneimine with a fatty acid. This amidation is described in Rannard and Davies (Organic Letters 2002, 2, 2117), and also in U.S. Pat. No. 3,425,549. The polyalkyleneimine is preferably polyethyleneimine or polypropyleneimine, in particular polyethyleneimine.

In one particular embodiment of the process, the hybrid complex material, in particular in the form of a solution, is applied from the outside to the product. The hybrid complex material here may be applied to the finished medical-technology product, in particular by spray-application or by immersion. The hybrid complex material is advantageously to be processed at room temperature, and is dried after application to the product.

The hybrid complex material is particularly preferably applied to a suture material, being applied to the suture material together with a lubricant, in particular applied in the form of a solution in an organic solvent, such as ethyl acetate.

In another embodiment, the hybrid complex material for producing medical-technology products is directly added to the polymer during the production of the product, in particular in the form of a solution. Addition to the material used to produce the product achieves uniform distribution of the biocidic hybrid complex material within the medical-technology product. This is in particular of decisive importance for resorbable or partially resorbable products, in order that, after the resorption of the surface layer of the product material, each further layer lying thereunder has the same biocidic properties and thus the entire surface of the entire product material has the antimicrobial properties over the lifetime of the product material.

In one advantageous embodiment, the hybrid complex material is mixed with the material used to produce the product, and is then moulded, in particular extruded, spun, pressed, rolled, cast or blown, to give the desired product. The mixture of polymer and hybrid complex is particularly preferably spun to give a thread material which, depending on the nature of the polymer used, is knitted or woven to give either resorbable or non-resorbable suture material or to give textile products.

The independent and dependent patent claims are hereby incorporated into the Description by way of reference.

Further features of the invention are apparent from the following description of preferred embodiments and examples. The individual features of the invention here may be realized alone or in combination with one another. The embodiments described serve for illustration and to improve understanding of the invention, and are in no way to be understood as limiting.

### DETAILED DESCRIPTION OF THE PREFERRED EMBODIMENTS

Starting from commercially available polyethyleneimine (PEI), the amphiphilic amidated polyethyleneimine (amPEI) is produced by amidation as in Rannard and Davies (Org. Let., 2000, 2, 2117).

The amPEI is dissolved in dry toluene. Silver nitrate is then dissolved in the toluene solution in a ratio of 0.5 (Ag⁺/N atoms). Reduction with Li[HBEt₃] gave a clear yellow colloidal silver solution. Complete reduction of Ag⁺ to Ag takes place via extraction of the colloidal solution with an aqueous solution of sodium thiosulphate. The reaction solution is then checked, using sodium sulphide, for any residual Ag⁺. Transmission electron microscopy (TEM) showed silver nanoparticles whose diameter was from 1 to 2 nm.

Glass microscope slides were coated with the polymer-nanoparticle-hybrid solution by concentrating one droplet of the solution. The microscope slide was then washed with a PBS buffer (pH 7) for 2 hours. Escherichia coli cells were applied to the microscope slide by aerosol spraying, and cultivated overnight under agar growth medium. Counting of the bacterial colonies grown on that region of the microscope slide coated with the hybrid complex revealed at least 98% fewer bacterial colonies than in the surrounding untreated regions of the microscope slide.

Comparative test using the following combinations of reagents:

No antimicrobial activity of any kind was found with amPEI/AgNO₃, am-PEI/Li[HBEt₃] and amPEI under the same experimental conditions.

Comparison of untreated and amidated PEI:

A comparative experiment under conditions identical to those above for the amidated polyethyleneimine was carried out using an unmodified PEI. Antimicrobial activity was found with this for the PEI/silver complex prior to the PBS-buffer-washing step described above, but absolutely no antimicrobial activity was found after the washing step. This shows that the modification of the polyethyleneimine to give the amphiphilic complexing agent is necessary for adequate adhesion to the substrate.

### BRIEF DESCRIPTION OF THE FIGURES

FIG. 1: shows a hybrid complex (1, 2) absorbed on a surface (3).

### DETAILED DESCRIPTION OF THE FIGURES

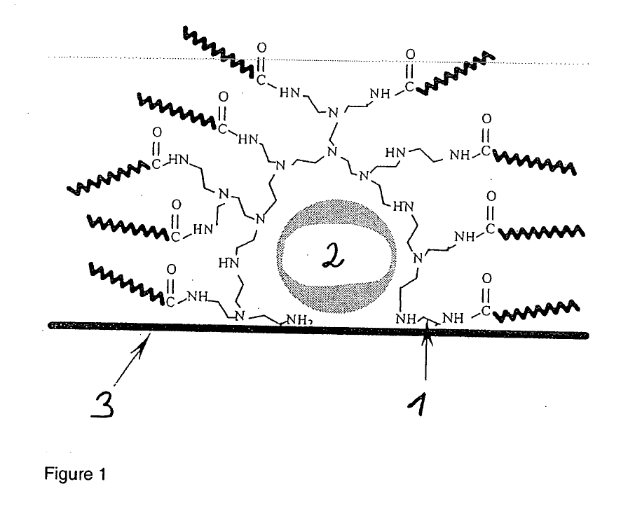

FIG. 1 shows a hybrid complex (1, 2) composed of a silver nanoparticle (2) in a capsule formed by a palmitic acid-amidated branched polyethyleneimine macro-molecule (1). The wavy lines here indicate a palmitic acid radical, i.e. a saturated hydrocarbon chain having 15 carbon atoms. Primary, secondary or tertiary nitrogen atoms directly surround the silver nanoparticle (2). Each of the palmitic acid radicals has been bonded to an amide group whose nitrogen atom derives from the polyethyleneimine skeleton, these previously representing primary amines, i.e. terminal NH₂ groups. Depending on the degree of branching of the polyethyleneimine, there is direct attachment of these amide groups to a nitrogen atom located immediately adjacent to the silver nanoparticle (2), or there is attachment of these amide groups to other secondary or tertiary nitrogen atoms not directly adjacent to the silver nanoparticle (2), the result being arrangements where the amide group with the attendant hydrophobic fatty acid radical is relatively near or relatively distant from the encapsulated silver nanoparticle (2) by virtue of these amine group spacers.

1. Medical-technology product with a layer of a hybrid complex material composed of a branched amphiphilic macromolecule and of a metal nanoparticle, the layer having been provided at least on the surface and on the surface at least on a portion of the surface.

2. Medical-technology product according to claim 1, wherein each metal nanoparticle has been surrounded in the manner of a capsule by at least one branched amphiphilic macromolecule.

3. Medical-technology product according to claim 1, wherein the amphiphilic macromolecule is an amphiphilic polyalkyleneimine.

4. Medical-technology product according to claim 1, wherein the amphiphilic macromolecule is an amphiphilic polyalkyleneimine with a degree of branching of 20% to 90%.

5. Medical-technology product according to claim 1, wherein the amphiphilic macromolecule is an amphiphilic polyalkyleneimine that has alkyl-substituted secondary or tertiary amino groups.

6. Medical-technology product according to claim 1, wherein the amphiphilic macromolecule is a branched amphiphilic polyalkyleneimine which has amide groups, where the N atoms of the amide groups derive from the polyalkyleneimine.

7. Medical-technology product according to claim 1, wherein the amphiphilic macromolecule is a branched amphiphilic polyalkyleneimine which has amide groups directed away from the metal nanoparticle and where the N atoms of the amide groups derive from the polyalkyleneimine.

8. Medical-technology product according to claim 6, wherein the amide groups have an aliphatic radical of a fatty acid having from 6 to 22 carbon atoms.

9. Medical-technology product according to claim 6, wherein the amide groups have an aliphatic radical of a fatty acid, oriented towards the outside, having from 6 to 22 carbon atoms.

10. Medical-technology product according to claim 1, wherein the molecular weight of the macromolecule is from 800 to 20 000.

11. Medical-technology product according to claim 1, wherein the metal nanoparticle is a silver nanoparticle or a copper nanoparticle.

12. Medical-technology product according to claim 3, wherein the metal nanoparticle is a silver nanoparticle with a ratio of silver atoms to nitrogen atoms in direct contact with the silver atoms is from 1:2 to 1:10.

13. Medical-technology product according to claim 1, wherein the diameter of the hybrid complex is from 0.5 to 10 nm.

14. Medical-technology product according to claim 1, wherein the diameter of the hybrid complex is about 2 nm.

15. Medical-technology product according to claim 1, wherein the product is a temporary or long-lasting implant for the body of a human or of an animal.

16. Medical-technology product according to claim 1, wherein the product is a medical instrument.

17. Medical-technology product according to claim 1, wherein the material of the product is metal.

18. Medical-technology product according to claim 1, wherein the material of the product is non-resorbable or at least to some extent resorbable polymers.

19. Medical-technology product according to claim 1, wherein the material of the product is ceramic.

20. Medical-technology product according to claim 1, wherein the product is sterilizable.

21. Medical-technology product with a biocide in the form of a hybrid complex material composed of a branched amphiphilic macromolecule and of a metal nanoparticle.

22. Medical-technology product according to claim 21, wherein the biocide has been applied to at least a portion of the surface of the medical-technology product.

23. Medical-technology product according to claim 21, wherein the biocide has been incorporated into the interior of the medical-technology product.

24. Medical-technology product according to claim 21, wherein the biocide has been applied to at least one portion of the surface and into the interior of the medical-technology product.

25. Medical-technology product according to claim 21, wherein each metal nanoparticle has been surrounded in the manner of a capsule by at least one branched amphiphilic macromolecule.

26. Process for producing a hybrid complex material composed of a branched amphiphilic macromolecule and of a metal nanoparticle by dissolving a metal compound in a solution of an amphiphilic polyalkyleneimine with complexing, followed by reduction of the metal compound.

27. Process according to claim 26, wherein each metal nanoparticle has been surrounded in the manner of a capsule by at least one branched amphiphilic macromolecule.

28. Process according to claim 26, wherein the metal compound is a silver salt.

29. Process for producing medical-technology products according to claim 1, wherein the hybrid complex material is applied from outside to the product.

30. Process for producing medical-technology products according to claim 1, wherein the hybrid complex material is applied from outside to the product in the form of a solution.

31. Process for producing medical-technology products according to claim 1, wherein the hybrid complex material is added to the polymer material of the product during its production.

32. Process according to claim 31, wherein the hybrid complex material is mixed and moulded with the material used to produce the product.

33. Process according to claim 31, wherein the hybrid complex material is mixed and spun with the material used to produce the product.
